(19) United States
(12) Patent Application Publication
(10) Pub. No.: US 2017/0267669 A1
(43) Pub. Date: Sep. 21, 2017

(54) Process for the Preparation of Rivaroxaban

(71) Applicant: Cipla Limited, Mumbai (IN)

(72) Inventors: Dharmaraj Ramachandra RAO, Mumbai (IN); Geena MALHOTRA, Mumbai (IN); Venkata Srinivas PULLELA, Bangalore (IN); Manohar Raghunath SURVE, Raigad (IN); Hemant Sanjeevan DORLE, Raigad (IN); Kiran Balasaheb JADHAV, Ahmednagar (IN)

(21) Appl. No.: 15/506,433

(22) PCT Filed: Aug. 24, 2015
(86) PCT No.: PCT/GB2015/052447

§ 371 Date: Feb. 24, 2017

(30) Foreign Application Priority Data
Aug. 25, 2014 (IN) 2722/MUM/2014

(51) Int. Cl. C07D 413/14; C07D 265/32; C07D 413/12; A61K 31/5377

(52) U.S. Cl. CPC C07D 413/14; A61K 31/5377; C07D 265/32; C07D 413/12

(57) ABSTRACT

The present invention relates to an environmentally friendly process for preparing rivaroxaban. The present invention provides a process for preparing rivaroxaban of formula I, the process comprising: reacting a compound of formula VI with a base in the presence of a solvent to form a compound of formula VII; and condensing the compound of formula VII with a compound of formula VIII or a compound of formula IX in the presence of a solvent to prepare rivaroxaban of formula I, wherein the solvent used in both steps comprises water.

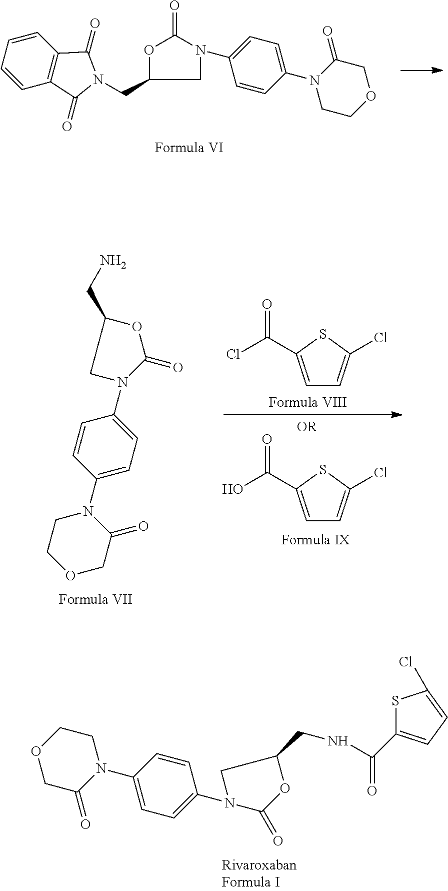 [SMILES: O=C1c2ccccc2C(=O)N1C[C@@H]1CN(c2ccc(N3CCOCC3=O)cc2)C(=O)O1 ; NC[C@@H]1CN(c2ccc(N3CCOCC3=O)cc2)C(=O)O1 ; O=C(Cl)c1ccc(Cl)s1 ; O=C(O)c1ccc(Cl)s1 ; O=C(NC[C@@H]1CN(c2ccc(N3CCOCC3=O)cc2)C(=O)O1)c1ccc(Cl)s1]

### CROSS REFERENCE TO RELATED APPLICATIONS

This application is a filing under 35 U.S.C. 371 of International Application No. PCT/GB2015/052447 filed Aug. 24, 2015, entitled “Process of the Preparation of Rivaroxaban” which claims priority to Indian Patent Application No. 2722/MUM/2014 filed Aug. 25, 2014, which applications are incorporated by reference herein in their entirety.

### FIELD OF INVENTION

The present invention relates to a novel, commercially viable and industrially advantageous process for the preparation of rivaroxaban, providing high yield and purity.

The present invention also provides novel process for the preparation of intermediates of rivaroxaban.

### BACKGROUND OF INVENTION

Rivaroxaban chemically is 5-chloro-N-({(5S)-2-oxo-3-[4-(3-oxo-4-morpholinyl) phenyl]-1,3-oxazolidin-5-yl} methyl)-2-thiophenecarboxamide of Formula I.

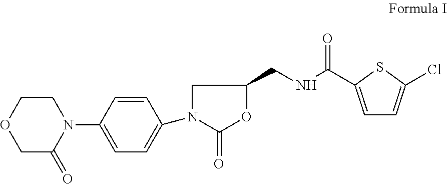 [SMILES: O=C(NC[C@@H]1CN(c2ccc(N3CCOCC3=O)cc2)C(=O)O1)c1ccc(Cl)s1]

Rivaroxaban is an oral anticoagulant. It is sold by Bayer under the brand name Xarelto® and it is orally administered as tablets containing 10 mg, 15 mg, and 20 mg of rivaroxaban.

U.S. Pat. No. 7,157,456 (hereinafter referred to as the '456 patent) discloses process for the preparation of rivaroxaban which comprises of reducing 4-(4-morpholin-3-onyl)nitrobenzene by hydrogenation to 4-(4-aminophenyl)-3-morpholinone using 5% Pd/C in tetrahydrofuran at 70° C. for 8 hours.

2-[(2S)-2-oxiranylmethyl]-1H-isoindole-1,3(2H)-dione is reacted with 4-(4-aminophenyl)-3-morpholinone in ethanol/water to give 2-((2R)-2-Hydroxy-3-{[4-(3-oxo-4-morpholinyl)phenyl] amino} propyl)-1H-isoindole-1,3(2H)-dione. Subsequently 2-((2R)-2-Hydroxy-3-{[4-(3-oxo-4-morpholinyl)phenyl]amino} propyl)-1H-isoindole-1,3(2H)-dione is cyclized with N,N′-carbonyldiimidazole in tetrahydrofuran to give 2-({(5S)-2-Oxo-3-[4-(3-oxo-4-morpholinyl)phenyl]-1,3-oxazolidin-5-yl}methyl)-1H-isoindole-1,3(2H)-dione. Phthalimide protective group is deprotected using methylamine (40% strength in water) in ethanol, which is reacted with 5-chlorothiophene-2-carbonyl chloride in pyridine.

The process for the preparation of rivaroxaban disclosed in '456 patent makes the use of toxic tetrahydrofuran, diethyl ether and carcinogenic pyridine as a solvent in large volume and hence may not be safe and economical. Reduction is also carried out in autoclave by hydrogenation at high temperature, elevated pressure and the reaction time is also prolonged. Pd/C is an expensive reagent and hence would add to the cost. Purity of intermediates and final product is not disclosed in this patent.

U.S. Pat. No. 7,598,378 provides a process for preparing 4-(4-aminophenyl)-3-morpholinone by reacting 4-(4-nitrophenyl)-3-morpholinone with hydrogen in the presence of a hydrogenation catalyst at 80° C. for one hour in an aliphatic alcohol.

U.S. Pat. No. 8,106,192 (hereinafter referred to as the '192 patent) provides a process for the preparation of N—((S)-3-bromo-2-hydroxypropyl)-5-chlorothiophene-2-carboxamide, wherein (2S)-3-aminopropane-1,2-diol hydrochloride is reacted with 5-chlorothiophene-2-carbonyl chloride in water:2-methyltetrahydrofuran mixture to provide N—((S)-2,3-dihydroxypropyl)-5-chlorothiophene-2-carboxamide.

The resulting compound is treated with hydrobromic acid (33% solution of hydrobromic acid in acetic acid) in the presence of acetic anhydride at 60° C. to 65° C. in methanol, and the reaction mixture is stirred overnight to give N—((S)-3-bromo-2-hydroxypropyl)-5-chlorothiophene-2-carboxamide, which is further converted into N—{(R)-2-hydroxy-3-[4-(3-oxomorpholin-4-yl)phenyl amino]propyl}-5-chlorothiophene-2-carboxamide in toluene at 103° C. to 105° C. for 6 hours, which is further treated with N,N-carbonyldiimidazole in the presence of 1-methyl-2-pyrrolidone and toluene at 115° C. for 1 hour into rivaroxaban.

The above processes involve the use of gases and solvents which may cause handling concerns or may be toxic or highly flammable.

U.S. Pat. No. 7,351,823 discloses a process for preparing rivaroxaban, in three steps. According to the first step, 5-chlorothiophene-2-carbonyl chloride is prepared by reacting 5-chlorothiophene-2-carboxylic acid with thionyl chloride in toluene at a temperature of 75° C. to 80° C.

According to the second step, 4-[4-[(5S)-5-(aminomethyl)-2-oxo-1,3-oxazolidin-3-yl]phenyl]morpholine-3-one hydrochloride salt is reacted with 5-chlorothiophene-2-carbonyl chloride (30% strength solution in toluene) in the presence of sodium carbonate in a solvent mixture containing water and acetone to produce crude rivaroxaban.

In the third step, the solvent-containing crude product is purified by recrystallization from acetic acid.

The processes for the preparation of rivaroxaban described in the aforementioned prior art involve many steps and suffer from disadvantages such as the use of highly hazardous materials like thionyl chloride, recrystallization using corrosive acids like acetic acid, toxic solvents like toluene, acetone which would in turn affect the product yield and quality, thereby making the process commercially less feasible.

The prior art processes disclosed above involve the use of harmful chemicals, gases or solvents which would not be safe for handling and would add to the environment pollution and hazards. Prior art processes also involve many steps which are complex thus making the process less feasible on a commercial scale and thus resource and time consuming. Based on these drawbacks, the prior art processes have been found to be unsuitable for the preparation of rivaroxaban on lab scale as well as on commercial scale operations.

Therefore, there is a need to develop an improved, commercially viable and environment friendly process for preparing rivaroxaban with high yield and purity using non-hazardous conditions, environment friendly and easy to handle reagents and solvents.

### SUMMARY OF THE INVENTION

Provided herein is an improved process for the preparation of rivaroxaban of formula I;

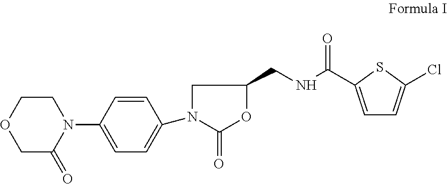 [SMILES: O=C(NC[C@@H]1CN(c2ccc(N3CCOCC3=O)cc2)C(=O)O1)c1ccc(Cl)s1]

According to one aspect of the present invention, there is provided a process for preparing rivaroxaban of formula I, the process comprising: reacting a compound of formula VI with a first base in the presence of a solvent to form a compound of formula VII; and condensing the compound of formula VII with a compound of formula VIII or a compound of formula IX in the presence of a second base and a solvent to prepare rivaroxaban of formula I

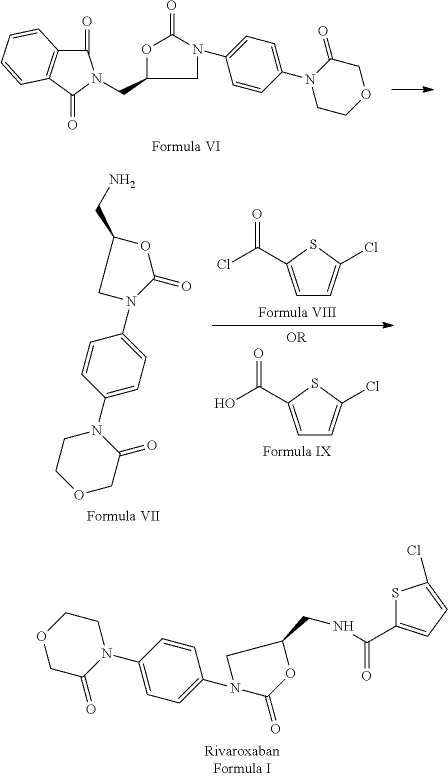 [SMILES: O=C1c2ccccc2C(=O)N1C[C@@H]1CN(c2ccc(N3CCOCC3=O)cc2)C(=O)O1 ; NC[C@@H]1CN(c2ccc(N3CCOCC3=O)cc2)C(=O)O1 ; O=C(Cl)c1ccc(Cl)s1 ; O=C(O)c1ccc(Cl)s1 ; O=C(NC[C@@H]1CN(c2ccc(N3CCOCC3=O)cc2)C(=O)O1)c1ccc(Cl)s1]

wherein the solvent used in both steps comprises water.

Suitably, the compound of formula VII is condensed with a compound of formula VIII in the presence of a solvent to prepare rivaroxaban of formula I. Alternatively, the compound of formula VII is condensed with or a compound of formula IX in the presence of a solvent to prepare rivaroxaban of formula I.

The solvent used in the step of forming compound of formula VII may be water alone (i.e. no other solvents being present). The solvent used in the step of forming rivaroxaban may be a mixture of water and one or more solvents. Preferably, the solvent used in the step of forming compound of formula VII is water alone and the solvent used in the step of forming rivaroxaban is a mixture of water and one or more solvents. The or each other solvent may be selected from the group consisting of acetone and dimethyl carbonate.

Preferably, the compound of formula VII is not isolated before the condensation step.

The first base may be selected from the group consisting of a C1-C4 alkyl ammonia; mono, a di or tri C1-C4 alkyl amine; a mono, di or tri hydroxy C1-C4 alkyl amine; morpholine; thiomorpholine; piperidine; N,N-dimethylaniline; pyridine; a hydrazine and pyrrolidine. The first base is preferably aqueous methyl amine.

The second base may be selected from the group consisting of an alkali or alkaline earth metal hydroxide; an alkoxide; a carbonate; a bicarbonate; and an organic base. The second base is preferably potassium carbonate.

The compound of formula VI may be prepared by cyclisation of a compound of formula V

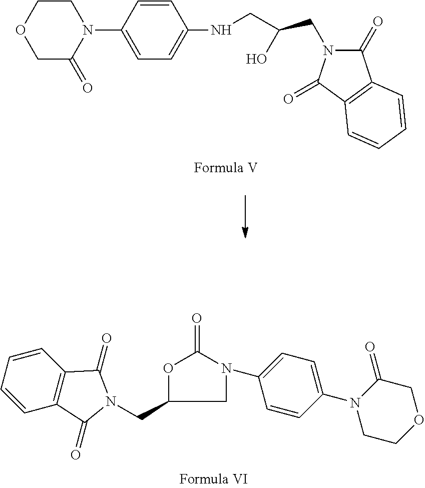 [SMILES: O=C1c2ccccc2C(=O)N1C[C@@H](O)CNc1ccc(N2CCOCC2=O)cc1 ; O=C1c2ccccc2C(=O)N1C[C@@H]1CN(c2ccc(N3CCOCC3=O)cc2)C(=O)O1]

in the presence of a carbonylating reagent and a base in a solvent which is a single solvent or a mixture of solvents.

The carbonylating reagent may be selected from the group consisting of N,N′-carbonyl diimidazole, N,N′-carbonyl-di-1,2,3-benzotriazole. N,N′-carbonyl-di-1,2,4-triazole, disuccinimidyl carbonate dialkyl carbonates, phosgene, diphosgene, triphosgene, methyl chloroformate, benzyl chloroformate and phenylchloroformate.

The base in the cyclisation step may be selected from one or more of an alkali or alkaline earth metal hydroxide; an alkoxide; a carbonate; a bicarbonate; and an organic base.

The solvent for the cyclisation step may be selected from the group consisting of an alcohol; a ketone; a dialkyl carbonate; a diaryl carbonates; and water.

The cyclisation reaction may be carried using dimethyl carbonate alone as the solvent, N,N′-carbonyldiimidazole as the carbonylating reagent and dimethylaminopyridine as the base.

The compound of formula VI may not be isolated before being reacted to form Compound VII. Alternatively, the compound of formula VI may be isolated before being reacted to form Compound VII.

The compound of formula V may be prepared by condensing a compound of formula III with (S)-Glycidyl Phthalimide (Formula IV);

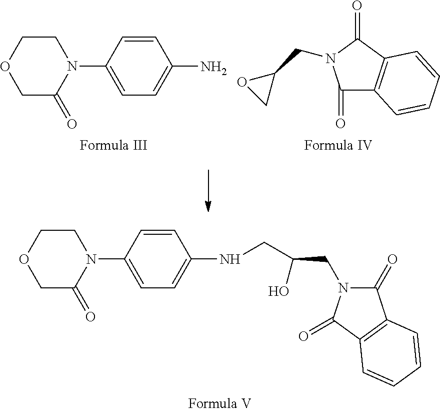

in the presence of a solvent which is a single solvent or a or mixture of solvents to form the compound of formula V.

The solvent for the condensation step to form compound V may be selected from the group consisting of a C₁-C₅ alcohol; a ketone; and water.

The condensation step to form compound V may be carried out using water alone as the solvent and without base.

The compound of formula V may not be isolated before being reacted to form compound VI. Alternatively, the compound of formula V is isolated before being reacted to form compound VI.

The compound of formula III may be prepared by reducing a compound of formula II

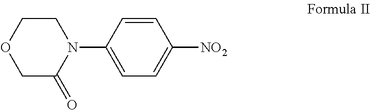 [SMILES: O=C1COCCN1c1ccc([N+](=O)[O-])cc1]

in the presence of a hydrogenation catalyst, and reagent and a solvent which is a single solvent or a or mixture of solvents to form the compound of formula III.

The solvent for the reduction may be selected from the group consisting of a C₁-C₃ alcohol, an ester, an ether, a nitrile, tetrahydrofuran, water, a halogenated solvent, dimethylformamide, dimethyl sulfoxide, sulfolane, or a mixture thereof.

The hydrogenation catalyst for the reduction may be selected from the group consisting of a noble metal catalyst which is preferably supported on a material selected from activated carbon, calcium carbonate, silicon dioxide, and triphenyl phosphine; a nickel-based catalyst selected from Raney nickel, and Urushibara nickel; zinc dust; palladium acetate; Iron; alumina; silica; calcium carbonate; barium sulphate; and a zeolite.

The reagent employed for reduction may be selected from formic acid, ammonium chloride, ammonium formate, ammonia, an alkaline metal hydride, an alkali metal hydride, hydrochloric acid, sodium hydrosulfide and ammonium sulfide.

The reduction step may involve the use of water alone as a solvent, Raney-Nickel as the catalyst and ammonium formate as the reagent.

The compound of formula III may not be isolated before being reacted to form compound V. Alternatively, the compound of formula III may be isolated before being reacted to form compound V.

According to another aspect of the present invention, there is provided an improved process for preparation of rivaroxaban of formula I;

which process comprises steps of:
a) Reducing 4-(4-nitrophenyl) morpholin-3-one (Formula II);

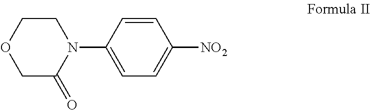 [SMILES: O=C1COCCN1c1ccc([N+](=O)[O-])cc1]

in a suitable solvent (either a single solvent or a or mixture of solvents) to obtain 4-(4-aminophenyl)morpholin-3-one (Formula III), suitably in the presence of a hydrogenation catalyst and a reagent;

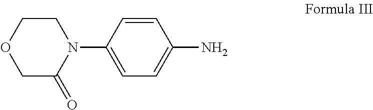 [SMILES: Nc1ccc(N2CCOCC2=O)cc1]

b) condensing compound (Formula III) with (S)-Glycidyl Phthalimide (Formula IV);

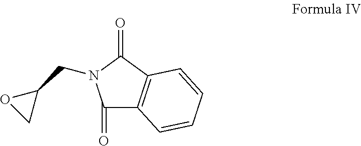 [SMILES: O=C1c2ccccc2C(=O)N1C[C@H]1CO1]

in a suitable solvent (either a single solvent or a or mixture of solvents) with or without base to obtain 2-((2R)-2-Hydroxy-3-{[4-(3-oxo-4-morpholinyl) phenyl] amino} propyl)-1H-isoindole-1,3(2H)-dione of formula V;

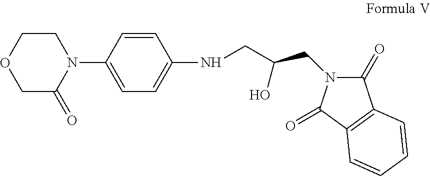 [SMILES: O=C1c2ccccc2C(=O)N1C[C@@H](O)CNc1ccc(N2CCOCC2=O)cc1]

c) cyclising 2-((2R)-2-Hydroxy-3-{[4-(3-oxo-4-morpholinyl)phenyl] amino} propyl)-1H-isoindole-1,3(2H)-dione of formula V in the presence of a carbonylating reagent and a base in a suitable solvent (either a single solvent or a or mixture of solvents) to obtain 2-({(5S)-2-Oxo-3-[4-(3-oxo-4-morpholinyl)phenyl]-1,3-oxazolidin-5-yl}methyl)-1H-isoindole-1,3(2H)-dione of formula VI;

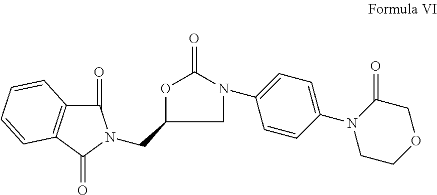 [SMILES: O=C1c2ccccc2C(=O)N1C[C@@H]1CN(c2ccc(N3CCOCC3=O)cc2)C(=O)O1]

d) deprotecting 2-({(5S)-2-Oxo-3-[4-(3-oxo-4-morpholinyl)phenyl]-1,3-oxazolidin-5-yl}methyl)-1H-isoindole-1,3(2H)-dione of formula VI in the presence of a base and a suitable solvent (either a single solvent or a or mixture of solvents) to obtain 4-[4-((S)-5-aminomethyl]-2-oxooxazolidin-3-yl) phenyl] morpholine-3-one of formula VII;

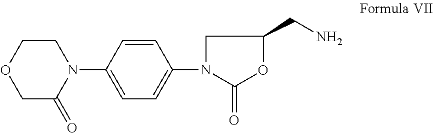 [SMILES: NC[C@@H]1CN(c2ccc(N3CCOCC3=O)cc2)C(=O)O1]

e) condensing 4-[4-((S)-5-aminomethyl]-2-oxooxazolidin-3-yl) phenyl] morpholine-3-one of formula VII with 5-chlorothiophene-2-carbonyl chloride of formula VIII or 5-chlorothiophene-2-carboxylic acid of formula IX;

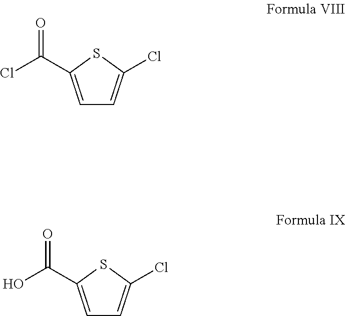 [SMILES: O=C(Cl)c1ccc(Cl)s1 ; O=C(O)c1ccc(Cl)s1]

in the presence of a suitable base and a suitable solvent (either a single solvent or a mixture of solvents) to obtain rivaroxaban of formula I.

Yet another aspect of the present invention relates to a process for the preparation of rivaroxaban of formula I without isolation of intermediates. Suitably, the steps may be the same as steps (a) to (e) above without isolation of the products of steps (a), (b), (c) and (d).

According to the first aspect of the present invention, there is provided a process for the preparation of rivaroxaban and its intermediates using an environmentally friendly solvent. Suitably, the steps may be the same as steps (a) to (e) above; with or without isolation of the intermediates; preferably without isolation.

According to another aspect of the present invention, there is provided a process for the preparation of rivaroxaban using an environmentally friendly solvent without isolation of intermediates.

Yet another aspect of the present invention, there is provided a process for the preparation of rivaroxaban (Formula I), wherein the said process eliminates toxic and hazardous solvents and reagents, use of hydrogen at high pressure, laborious workup, prolonged reaction time, high temperature, and extensive purifications and hence makes this process cost effective, efficient, and eco-friendly. Suitably, the steps may be the same as steps (a) to (e) above; with or without isolation of the intermediates; preferably without isolation.

According to another aspect of the present invention, there is provided a pharmaceutical composition comprising rivaroxaban prepared by a process as described above, together with one or more pharmaceutically acceptable excipients. Such excipients are well known to those skilled in the art.

Further aspect of the present invention provides use of rivaroxaban of formula obtained or obtainable by the process of the present invention for the manufacture of therapeutic agent.

In yet another aspect of the present invention there is provided use of rivaroxaban of formula I obtained or obtainable by the process of the present invention, in the treatment of deep vein thrombosis or pulmonary embolism and/or the prevention of recurrent venous thromboembolism.

In yet another aspect of the present invention there is provided use of rivaroxaban of formula I obtained or obtainable by the process of the present invention, in the manufacture of a medicament for the treatment of deep vein thrombosis or pulmonary embolism and/or for the prevention of recurrent venous thromboembolism.

In yet another aspect of the present invention there is provided a method for the treatment of deep vein thrombosis and/or pulmonary embolism and the prevention of recurrent venous thromboembolism, comprising administering the rivaroxaban of formula I obtained or obtainable by a process of the present invention.

### DETAILED DESCRIPTION OF THE INVENTION

The present invention provides a novel process for the preparation of 5-chloro-N-({(5S)-2-oxo-3-[4-(3-oxo-4-morpholinyl) phenyl]-1,3-oxazolidin-5-yl} methyl)-2-thiophene carboxamide of formula I.

Scheme I is a schematic representation of an example of the process for the preparation of Formula I.

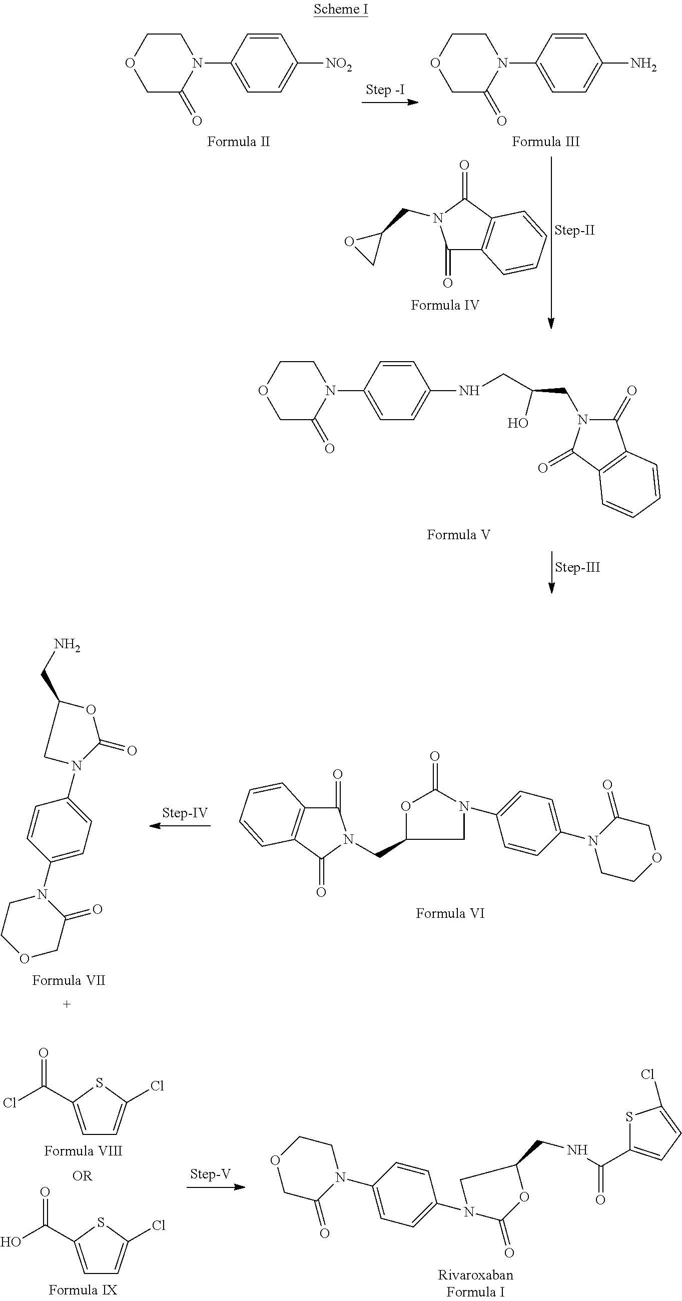

Each of steps I to IV form separate aspects of the invention, and each step may be combined with one or more other steps to form further aspects of the invention.

### Step I: Reduction of 4-(4-nitrophenyl) morpholin-3-one (Formula II) to 4-(4-aminophenyl)morpholin-3-one (Formula III)

In one aspect of the present invention 4-(4-nitrophenyl) morpholin-3-one (Formula II) is reduced to 4-(4-aminophenyl)morpholin-3-one (Formula III), in the presence of a hydrogenation catalyst and reagent (suitably at atmospheric pressure, i.e. at a pressure around (760 mm Hg/101325 Pa)) in a suitable solvent. The solvent may be a single solvent or a mixture of solvents.

The suitable solvent employed in step I may be selected from one or more of alcohols, esters, ethers, nitriles, tetrahydrofuran (THF), water, halogenated solvents, dimethylformamide, dimethyl sulfoxide, sulfolane, or a mixture thereof. The preferable solvent is water alone.

The hydrogenation catalyst employed in step I may be selected from a noble metal catalyst such as ruthenium, rhodium, palladium, silver, osmium, iridium, platinum and gold which may be supported on various materials such as activated carbon, calcium carbonate, silicon dioxide, triphenyl phosphine; nickel based catalyst such as Raney nickel, Urushibara nickel; zinc dust, palladium acetate, Iron, alumina, silica, calcium carbonate, barium sulphate, zeolites. The preferable catalyst is Raney-nickel.

The reagents employed for reduction may be selected from formic acid, ammonium chloride, ammonium formate, ammonia, alkaline metal hydrides, alkali metal hydrides, hydrochloric acid, sodium hydrosulfide or ammonium sulfide. The preferable reagent is ammonium formate.

The reduction step may involve the use of water alone as a solvent, Raney-Nickel as the catalyst and ammonium formate as the reagent.

The reaction in step I may be carried out at a temperature ranging from about 0° C. to about 200° C.; preferably from about 60° C. to about 120° C.; more preferably from about 80° C. to about 100° C. The preferable temperature is about 90° C.

According to another aspect, the present invention provides 4-(4-aminophenyl)morpholin-3-one (Formula III) prepared according to the above process.

The 4-(4-aminophenyl)morpholin-3-one (Formula III) prepared according to this process may be used to prepare rivaroxaban of formula I by any method, including any method disclosed herein. The 4-(4-nitrophenyl) morpholin-3-one (Formula II) used in the above process may have been prepared by any known process.

### Step II: condensing of 4-(4-aminophenyl)morpholin-3-one (Formula III) with (S)-Glycidyl Phthalimide (Formula IV) to form 2-((2R)-2-Hydroxy-3-{[4-(3-oxo-4-morpholinyl) phenyl]amino} propyl)-1H-isoindole-1,3(2H)-dione of formula V

Another aspect of the present invention is to provide a process for the preparation of 2-((2R)-2-Hydroxy-3-{[4-(3-oxo-4-morpholinyl) phenyl] amino} propyl)-1H-isoindole-1,3(2H)-dione of formula V, wherein the process comprises condensing compound (Formula III) with (S)-Glycidyl Phthalimide (Formula IV) in a suitable solvent, typically without a base. The solvent may be a single solvent or a mixture of solvents.

The suitable solvent employed for step II may be selected from the group consisting of C₁-C₅ alcohols such as methanol and ethanol, ketones such as acetone and water. The preferable solvent is water.

The reaction in step II may be carried out using water alone as the solvent and without base.

The reaction in step II may be carried out at a temperature ranging from about 0° C. to about 100° C. The preferable range is from about 70° C. to about 80° C.

After completion of reaction, the product may be washed with water.

Prior art suggests the use of 35 volumes of ethanol, and the isolated product is washed with diethyl ether, whereas in the present invention the reaction is performed using 35 volumes of water and the isolated product is washed with water. The use of water as a solvent makes the process feasible and safe to handle on the industrial scale. This forms an aspect of the invention.

Further, the use of water as solvent reduces the reaction time to 8 hours as compared to the prior art process which requires 27 hours. Thus this avoids the use of toxic and hazardous solvents and reduces prolonged reaction time. This forms another aspect of the invention.

According to another aspect, the present invention provides 2-((2R)-2-Hydroxy-3-{[4-(3-oxo-4-morpholinyl) phenyl] amino} propyl)-1H-isoindole-1,3(2H)-dione of formula V prepared according to the above process.

The 2-((2R)-2-Hydroxy-3-{[4-(3-oxo-4-morpholinyl) phenyl] amino} propyl)-1H-isoindole-1,3(2H)-dione of formula V prepared according to this process may be used to prepare rivaroxaban of formula I by any method, including any method disclosed herein. The 4-(4-aminophenyl)morpholin-3-one (Formula III) used in the above process may have been prepared by any process, including any process disclosed herein.

### Step III: cyclising 2-((2R)-2-Hydroxy-3-{[4-(3-oxo-4-morpholinyl)phenyl] amino}propyl)-1H-isoindole-1,3(2H)-dione of formula V to form 2-({(5S)-2-Oxo-3-[4-(3-oxo-4-morpholinyl)phenyl]-1,3-oxazolidin-5-yl}methyl)-1H-isoindole-1,3(2H)-dione of formula VI

Yet another aspect of the present invention provides a process for the preparation of 2-({(5S)-2-Oxo-3-[4-(3-oxo-4-morpholinyl)phenyl]-1,3-oxazolidin-5-yl}methyl)-1H-isoindole-1,3 (2H)-dione of formula VI, wherein the process comprises cyclisation of 2-((2R)-2-Hydroxy-3-{[4-(3-oxo-4-morpholinyl)phenyl] amino}propyl)-1H-isoindole-1,3(2H)-dione of formula V in the presence of a carbonylating reagent and a base in a suitable solvent. The solvent may be a single solvent or a mixture of solvents.

The carbonylating reagent in step III may be selected from the group consisting of N,N′-carbonyl diimidazole. N,N′-carbonyl-di-1,2,3-benzotriazole, N,N′-carbonyl-di-1,2,4-triazole, disuccinimidyl carbonate dialkyl carbonates, phosgene, diphosgene, triphosgene, methyl chloroformate, benzyl chloroformate and phenylchloroformate; the preferable carbonylating reagent is N,N′-carbonyldiimidazole.

The base in step III may be selected from one or more of alkali or alkaline earth metal hydroxides such as sodium hydroxide, potassium hydroxide; alkoxides such as sodium methoxide, potassium tert-butoxide; carbonates such as sodium carbonate, potassium carbonate; bicarbonates such as sodium bicarbonate, potassium bicarbonate; an organic base such as ammonia, triethylamine, diisopropylamine, dimethyl amine, Dimethylaminopyridine diisopropylethylamine, diisopropylmethylamine, pyridine, piperidine, morpholine and N-methyl piperidine. The preferable base is dimethylaminopyridine.

The suitable solvent in step III may be selected from the group consisting of alcohols such as methanol, ethanol; ketones such as acetone, dialkyl carbonates such as dimethyl carbonate, diethyl carbonate, di(n-propyl)carbonate, di(isopropyl)carbonate, di(n-butyl)carbonate, di(sec-butyl)carbonate, di(tert-butyl)carbonate or dihexyl carbonate, diaryl carbonates such as diphenyl carbonate, bis-methyl salicyl carbonate; water and like. Preferably the solvent is dimethyl carbonate.

The reaction in step III may be carried using dimethyl carbonate alone as the solvent, N,N′-carbonyldiimidazole as the carbonylating reagent and dimethylaminopyridine as the base.

The reaction in step III may be carried out at a temperature ranging from about 0° C. to about 100° C. Preferably ranging from about 60° C. to about 90° C.

According to another aspect, the present invention provides 2-({(5S)-2-Oxo-3-[4-(3-oxo-4-morpholinyl)phenyl]-1,3-oxazolidin-5-yl}methyl)-1H-isoindole-1,3(2H)-dione of formula VI

prepared according to the above process.

The 2-({(5S)-2-Oxo-3-[4-(3-oxo-4-morpholinyl)phenyl]-1,3-oxazolidin-5-yl}methyl)-1H-isoindole-1,3(2H)-dione of formula VI prepared according to this process may be used to prepare rivaroxaban of formula I by any method, including any method disclosed herein. The 2-((2R)-2-Hydroxy-3-{[4-(3-oxo-4-morpholinyl)phenyl] amino}propyl)-1H-isoindole-1,3(2H)-dione of formula V used in the above process may have been prepared by any process, including any process disclosed herein.

### Step IV: preparation of 4-[4-((S)-5-aminomethyl]-2-oxooxazolidin-3-yl) phenyl] morpholine-3-one of formula VII from 2-((5S)-2-Oxo-3-[4-(3-oxo-4-morpholinyl)phenyl]-1,3-oxazolidin-5-yl}methyl)-1H-isoindole-1,3(2H)-dione of formula VI

Yet another aspect of the present invention provides a process for the preparation of 4-[4-((S)-5-aminomethyl]-2-oxooxazolidin-3-yl) phenyl] morpholine-3-one of formula VII from 2-({(5S)-2-Oxo-3-[4-(3-oxo-4-morpholinyl)phenyl]-1,3-oxazolidin-5-yl}methyl)-1H-isoindole-1,3(2H)-dione of formula VI in the presence of a base and a suitable solvent. The solvent may be a single solvent or a mixture of solvents.

The base in step IV may be selected from the group consisting of a C1-C4 alkyl ammonia; mono, di or tri C1-C4 alkyl amine such as triethylamine, diisipropropyl ethyl amine; mono, di or tri hydroxy C1-C4 alkyl amine; morpholine; thiomorpholine; piperidine; N,N-dimethylaniline; pyridine; hydrazines and pyrrolidine. The preferred base is aqueous methyl amine; suitably employed as a 40% aqueous methyl amine solution.

The suitable solvent employed for step IV may be selected from the group consisting of C₁-C₅ alcohols such as methanol or ethanol, ketones such as acetone, water and mixtures thereof. The preferable solvent is water.

The reaction in step IV may be carried out using aqueous methyl amine as the base and water alone as the solvent.

The reaction in step IV may be carried out at a temperature ranging from about 25° C. to about 50° C. Preferably at a temperature of about 35° C.

According to another aspect, the present invention provides 4-[4-((S)-5-aminomethyl]-2-oxooxazolidin-3-yl) phenyl] morpholine-3-one of formula VII prepared according to the above process.

The 4-[4-((S)-5-aminomethyl]-2-oxooxazolidin-3-yl) phenyl] morpholine-3-one of formula VII prepared according to this process may be used to prepare rivaroxaban of formula I by any method, including any method disclosed herein. The 2-({(5S)-2-Oxo-3-[4-(3-oxo-4-morpholinyl)phenyl]-1,3-oxazolidin-5-yl}methyl)-1H-isoindole-1,3(2H)-dione of formula VI used in the above process may have been prepared by any process, including any process disclosed herein.

### Step V: condensing 4-[4-(S)-5-aminomethyl]-2-oxooxazolidin-3-yl) phenyl] morpholine-3-one of formula VII with 5-chlorothiophene-2-carbonyl chloride of formula VIII or with 5-chlorothiophene-2-carboxylic acid of formula IX

Yet another aspect of the present invention provides a process comprising condensing 4-[4-((S)-5-aminomethyl]-2-oxooxazolidin-3-yl) phenyl] morpholine-3-one of formula VII with 5-chlorothiophene-2-carbonyl chloride of formula VIII in the presence of a suitable base and a suitable solvent to obtain rivaroxaban of formula I.

Yet another aspect of the present invention provides a process comprising condensing 4-[4-((S)-5-aminomethyl]-2-oxooxazolidin-3-yl) phenyl] morpholine-3-one of formula VII with 5-chlorothiophene-2-carboxylic acid of formula IX in the presence of a suitable base in a suitable solvent (either a single solvent or a or mixture of solvents) to obtain rivaroxaban of formula I. Typically, the reaction is carried out using a suitable coupling agent.

The suitable coupling agent employed (when the process comprises condensing 4-[4-((S)-5-aminomethyl]-2-oxooxazolidin-3-yl) phenyl] morpholine-3-one of formula VII with 5-chlorothiophene-2-carboxylic acid of formula IX) may be selected from the group consisting of dicyclohexylcarbodiimide (DCC), N, N′-diisopropylcarbodiimide (DIC), 1-(3-dimethylaminopropyl)-3-ethylcarbodiimide hydrochloride (EDAC), 1-(3-dimethylaminopropyl)-3-ethylcarbodiimide (EDC), N-tert-butyl-N′-methylcarbodiimide (TBMC), and N-tert-butyl-N′-ethylcarbodiimide (TBEC), 1,1′-Carbonyldiimidazole (CDI), and triphosgene.

The base in step V may be selected from the group consisting of one or more of alkali or alkaline earth metal hydroxides such as sodium hydroxide, potassium hydroxide, magnesium hydroxide, barium hydroxide; alkoxides such as sodium methoxide, potassium tert-butoxide; carbonates such as sodium carbonate, potassium carbonate; bicarbonates such as sodium bicarbonate, potassium bicarbonate; an organic base such as triethylamine, diisopropylamine, dimethyl amine, diisopropylethylamine, diisopropylmethylamine, pyridine, piperidine, morpholine and N-methyl piperidine. The preferable base is potassium carbonate.

The suitable solvent in step V is selected from the group consisting of alcohols such as methanol, ethanol; ketones such as acetone, dialkyl carbonates such as dimethyl carbonate, diethyl carbonate, di(n-propyl)carbonate, di(isopropyl)carbonate, di(n-butyl)carbonate, di(sec-butyl)carbonate, di(tert-butyl)carbonate or dihexyl carbonate, diaryl carbonates such as diphenyl carbonate, bis-methyl salicyl carbonate; water and mixtures thereof: The referable solvent mixture is water and dimethyl carbonate.

The reaction in step V may be carried out using a carbonate as the base (preferably potassium carbonate) and a solvent mixture of water and dimethyl carbonate.

The reaction in step V may be carried out at a temperature ranging from about 0° C. to about 150° C.; preferably from about 0° C. to about 50° C.; more preferably from about 5° C. to about 20° C. Most preferably, the temperature ranges from about 10° C. to about 15° C.

The 4-[4-((S)-5-aminomethyl]-2-oxooxazolidin-3-yl) phenyl] morpholine-3-one of formula VII used in the above process may have been prepared by any process, including any process disclosed herein. The 5-chlorothiophene-2-carbonyl chloride of formula VIII and the 5-chlorothiophene-2-carboxylic acid of formula IX may be produced according to any known method.

Preparation of Rivaroxaban of Formula I without Isolation of Intermediates

Yet another aspect of the present invention is to provide a process for the preparation of rivaroxaban of formula I without isolation of intermediates (also referred to as “in one pot” and other related variations) as depicted in Scheme II.

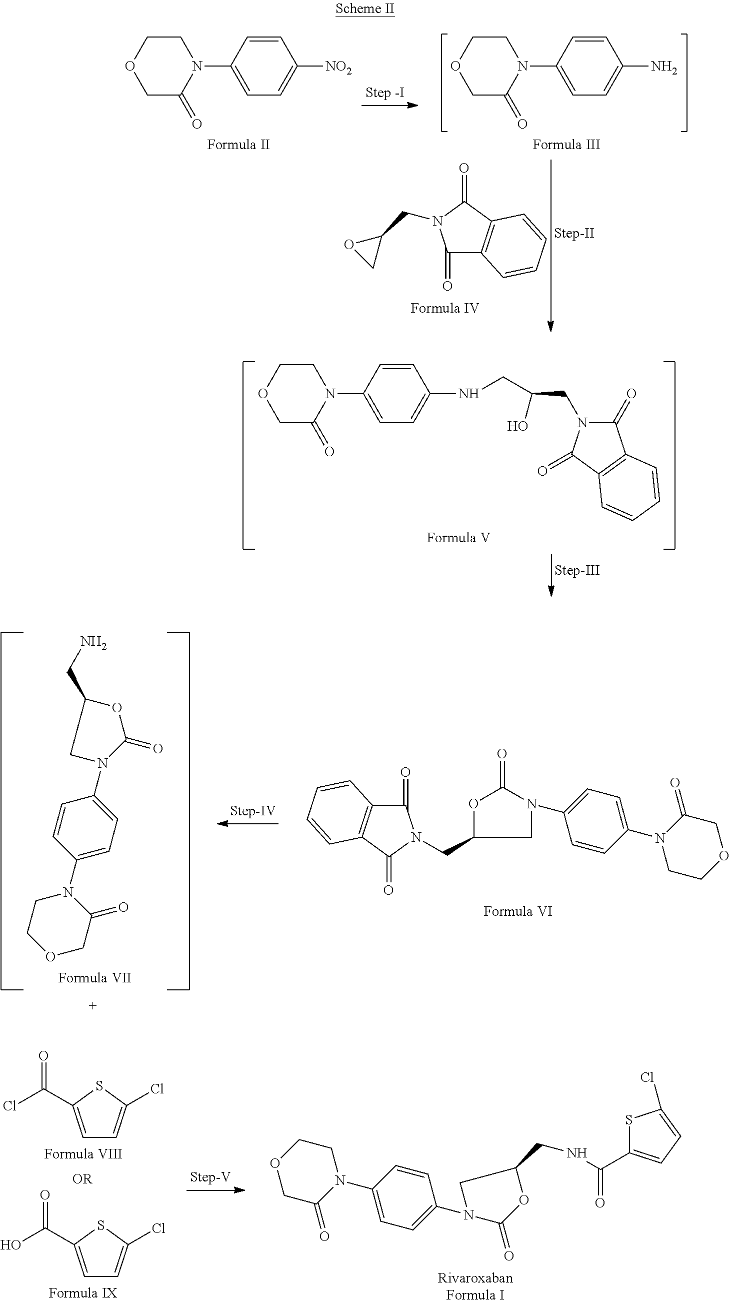

As used herein. “[ ]”/bracket in Scheme II indicates the intermediates are not isolated in the synthesis of rivaroxaban of formula I.

The reagents, solvents and reaction conditions employed may be similar or identical to those employed in the reactions as depicted in Scheme I.

Suitably, the reaction comprises:

(i) preparing a compound of formula III by reducing a compound of formula II

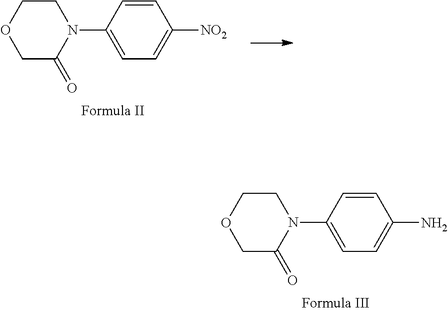 [SMILES: O=C1COCCN1c1ccc([N+](=O)[O-])cc1 ; Nc1ccc(N2CCOCC2=O)cc1]

in the presence of a hydrogenation catalyst, and reagent and a solvent which is a single solvent or a or mixture of solvents to form the compound of formula III; without isolating the compound of formula III, using it in step (ii);
(ii) preparing a compound of formula V by condensing the compound of formula III with (S)-Glycidyl Phthalimide (Formula IV);

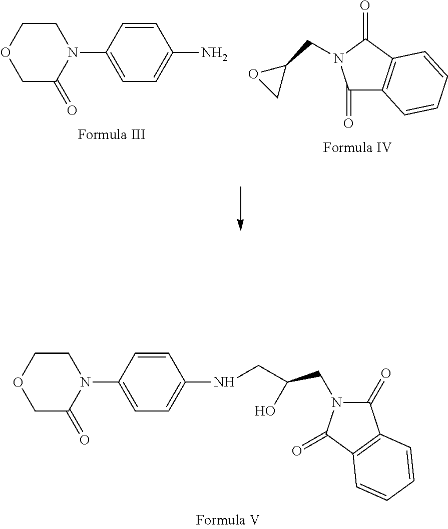 [SMILES: Nc1ccc(N2CCOCC2=O)cc1 ; O=C1c2ccccc2C(=O)N1C[C@H]1CO1 ; O=C1c2ccccc2C(=O)N1C[C@@H](O)CNc1ccc(N2CCOCC2=O)cc1]

in the presence of a solvent which is a single solvent or a or mixture of solvents to form the compound of formula V; without isolating the compound of formula V, using it in step (iii);
(iii) preparing a compound of formula VI by cyclisation of the compound of formula V

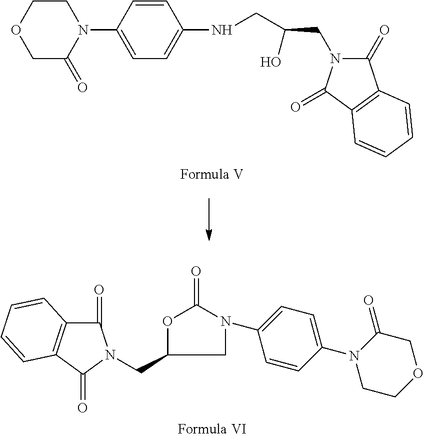 [SMILES: O=C1c2ccccc2C(=O)N1C[C@@H](O)CNc1ccc(N2CCOCC2=O)cc1 ; O=C1c2ccccc2C(=O)N1C[C@@H]1CN(c2ccc(N3CCOCC3=O)cc2)C(=O)O1]

in the presence of a carbonylating reagent and a base in a solvent which is a single solvent or a mixture of solvents; without isolating the compound of formula VI, using it in step (iv);
(iv) reacting the compound of formula VI with a base in the presence of a solvent to form a compound of formula VII; without isolating the compound of formula VII, using it in step (v); and
(v) condensing the compound of formula VII with a compound of formula VIII or a compound of formula IX in the presence of a solvent to prepare rivaroxaban of formula I

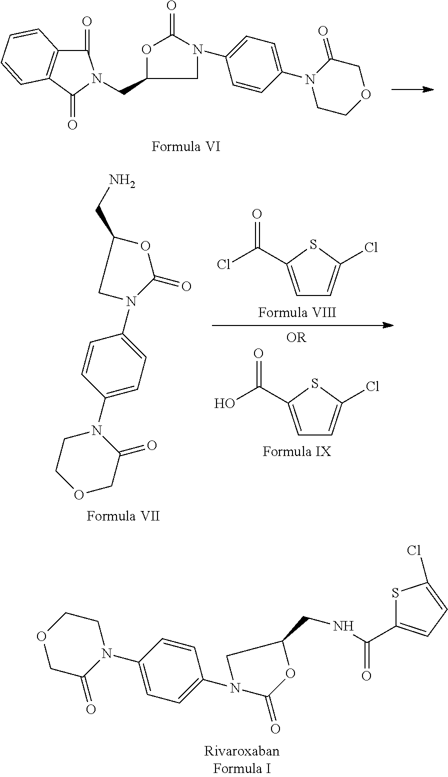 [SMILES: O=C1c2ccccc2C(=O)N1C[C@@H]1CN(c2ccc(N3CCOCC3=O)cc2)C(=O)O1 ; NC[C@@H]1CN(c2ccc(N3CCOCC3=O)cc2)C(=O)O1 ; O=C(Cl)c1ccc(Cl)s1 ; O=C(O)c1ccc(Cl)s1 ; O=C(NC[C@@H]1CN(c2ccc(N3CCOCC3=O)cc2)C(=O)O1)c1ccc(Cl)s1]

Suitably, the reduction step (i) involves the use of water alone as a solvent, Raney-Nickel as the catalyst and ammonium formate as the reagent. Suitably, the reaction in step (ii) is carried out using water alone as the solvent and without base. Suitably, the reaction in step III is carried using dimethyl carbonate alone as the solvent, N,N′-carbonyldiimidazole as the carbonylating reagent and dimethylaminopyridine as the base. Suitably, the solvent used in both steps (iv) and (v) comprises water.

One-pot synthesis (i.e. without isolation of the intermediates III, V, VI and VII) avoids lengthy separation process such as filtration, washing and purification of the intermediates and also saves time and resources thus increasing the chemical yield and making the process economical and suitable for industrial scale up.

The present invention is described in further detail below through examples, which however are provided only for illustrative purposes and the invention is in no way limited thereto.

### EXAMPLES

### Example 1

### Step I: 4-(4-Aminophenyl) morpholin-3-one (III)

To a reaction mixture of 100 gm (0.45 moles) 4-(4-Nitrophenyl) morpholin-3-one (II) in 1000 ml of purified water, 30 gm Raney Nickel and 850 gm (13.49 moles) ammonium formate were charged at 30° C. The reaction mixture was heated to 90° C. After completion of reaction 200 ml dichloromethane was added to reaction mixture. The reaction mixture was filtered through hyflo bed and washed with 200 ml purified water and further extracted with dichloromethane. Dichloromethane was distilled under vacuum. 500 ml ethyl acetate was charged to the reaction mixture and stirred for 1 hour at 0° C. to 5° C. The product was filtered and dried under vacuum at 50° C. to 55° C. for 10-12 hours.

Dried weight=75 gm

### Example 2

### Step II: 2-((2R)-2-Hydroxy-3-{[4-(3-oxo-4-morpholinyl) phenyl] amino} propyl)-1H-isoindole-1,3(2H)-dione (V)

To 100 gm (0.5208 moles) of 4-(4-Aminophenyl) morpholin-3-one (III) in 2500 ml purified water, 185 gm (0.9104 moles) of (S)-Glycidyl Phthalimide (IV) and 1000 ml purified water was charged and the reaction mixture was heated to 80° C. for 8 hours. The precipitated products was filtered and washed with purified water. The product was dried under vacuum at 60° C. to 65° C. for 24 hours.

Dried weight=203 gm

### Example 3

### Step III: 2-({(5S)-2-Oxo-3-[4-(3-oxo-4-morpholinyl)phenyl]-1,3-oxazolidin-5-yl}methyl)-1H-isoindole-1,3(2H)-dione (VI)

100 gm (0.2531 moles) 2-((2R)-2-Hydroxy-3-{[4-(3-oxo-4-morpholinyl) phenyl]amino} propyl)-1H-isoindole-1,3(2H)-dione (V) is added in 700 ml Dimethyl carbonate, 69.9 gm (0.4301 moles) Carbonyl diimidazole and 15.4 gm (0.1260 moles) Dimethylaminopyridine. Reaction mass is heated to 80-85° C. for 6 hours. It is then cooled, chilled and filtered to get 2-({(5S)-2-Oxo-3-[4-(3-oxo-4-morpholinyl)phenyl]-1,3-oxazolidin-5-yl}methyl)-1H-isoindole-1,3(2H)-dione. It is washed with dimethyl carbonate followed by water.

### Wet Wt=95 gm

### Example 4

### Step IV: 4-[4-((S)-5-aminomethyl]-2-oxooxazolidin-3-yl)phenyl]morpholine-3-one (VII)

100 gm (wet) (0.2375 moles) 2-({(5S)-2-Oxo-3-[4-(3-oxo-4-morpholinyl)phenyl]-1,3-oxazolidin-5-yl}methyl)-1H-isoindole-1,3(2H)-dione (VI) is added in 500 ml water and (46.02 gm, 1.4845 moles) methyl amine solution (40% Aqueous). Reaction mass is stirred at 35° C. for 5 hours to get 4-[4-((S)-5-aminomethyl]-2-oxooxazolidin-3-yl) phenyl]morpholine-3-one (VII). Reaction mass is subjected for degassing (i. e. kept under vacuum) for 1 hour to remove excess methyl amine.

This aqueous reaction mass is used as such for the preparation of Rivaroxaban without isolation.

### Example 5

### 5-chloro thiophene-2-carbonyl chloride (VIII)

62 gm (0.3827 moles) 5-Chloro thiophene-2-carboxylic acid was charged in 310 ml toluene. To it was charged 67 gm (0.5630 moles) of thionyl chloride at room temperature. The reaction mixture was heated to reflux temperature for about 10-12 hours. Toluene was distilled out completely to get an oil of 5-chloro thiophene-2-carbonyl chloride (68 gm).

### Example: 6

### Step V: with 5-chloro thiophene-2-carbonyl chloride (VIII) 5-chloro-N-({(5S)-2-oxo-3-[14-(3-oxo-4-morpholinyl) phenyl]-1,3-oxazolidin-5-yl} methyl)-2-thiophenecarboxamide (Rivaroxaban)

Aqueous reaction mass of example 4 containing 4-[4-((S)-5-aminomethyl]-2-oxooxazolidin-3-yl)phenyl]morpholine-3-one (VII) is chilled up to 10-15° C. Charged 49.16 gm (0.3562 moles) potassium carbonate at 10-15° C. The solution of 5-chloro thiophene-2-carbonyl chloride (VIII) 51.6 gm (0.2850 moles) in 200 ml dimethyl carbonate was charged to the reaction mixture at 10-15° C. Precipitated reaction mixture was stirred at 10-15° C. for 4 hours. The product was filtered and washed with water. The product was dried under vacuum at 65° C. It is then further purified by methanol purification at reflux temperature followed by DMSO/MeOH purification to get pure Rivaroxaban.

Dried weight=65.6 gm

### Example 7

### Synthesis of Rivaroxaban (One Pot Process)

To a reaction mixture of 100 gm (0.45 moles) 4-(4-Nitrophenyl) morpholin-3-one in 1000 ml of purified water, charged 30 gm Raney nickel and 850 gm (13.49 moles) ammonium formate lot wise at 30° C. The reaction mixture was heated to 90° C. Reaction mixture was cooled to 25-30° C. The reaction mixture was filtered and the filtrate was used in the next step.

To the filtrate, charged 185 gm (0.9104 moles) (S)-Glycidyl Phthalimide. The reaction mixture was heated to 70° C. for 10 hours. The reaction mixture was cooled to room temperature and taken for next stage.

196.49 gm (0.6615 moles) triphosgene and 507.16 ml (3.6455 moles) of triethylamine were charged to above reaction mixture at 0° C. to 5° C. The reaction mixture was heated to 70° C. for 8 hours. The reaction mixture was cooled to 0° C. to 50° C. 253.58 ml (1.8227 moles) of triethylamine was charged and heated to 70° C. for 7 hours. The reaction mixture was cooled to 0° C. to 5° C. Inorganics formed during reaction were filtered through the hyflo bed. Filtrate taken for next stage.

The filtrate was cooled to 5° C. to 10° C. and charged with 38.8 gm (0.3007 moles) N,N-diisopropylethylamine and 146 ml acetone. A solution of 5-chloro thiophene-2-carbonyl chloride 68 gm (0.3757 moles) in dimethyl carbonate was added at 5° C. to 10° C. The reaction mixture was heated to 20° C. to 25° C. for one hour. Then the reaction mixture was heated to 50° C. to 55° C. for one hour. The reaction mixture was cooled to 20-25° C. for one hour. The product rivaroxaban was filtered and washed with water and dried at 45° C. under vacuum for 8-10 hours.

Dried weight=87.6 gm

It will be appreciated that the invention may be modified within the scope of the appended claims.

1. A process for preparing rivaroxaban of formula I, the process comprising: reacting a compound of formula VI with a first base in the presence of a solvent to form a compound of formula VII; and condensing the compound of formula VII with a compound of formula VIII or a compound of formula IX with a second base in the presence of a solvent to prepare rivaroxaban of formula I 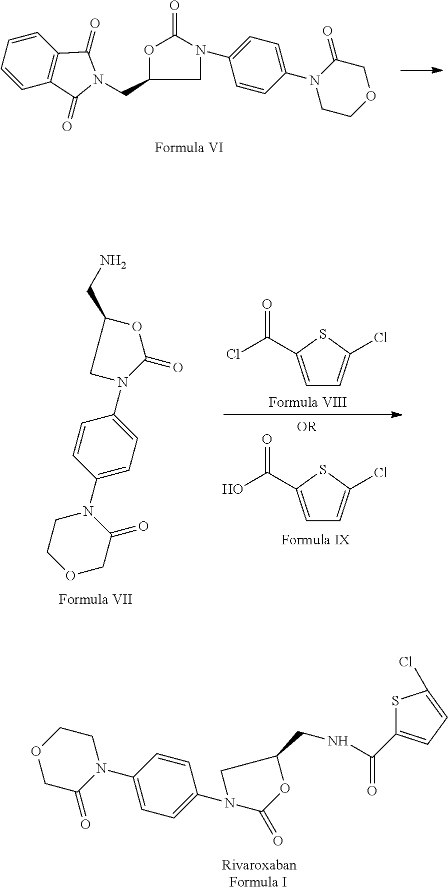 [SMILES: O=C1c2ccccc2C(=O)N1C[C@@H]1CN(c2ccc(N3CCOCC3=O)cc2)C(=O)O1 ; NC[C@@H]1CN(c2ccc(N3CCOCC3=O)cc2)C(=O)O1 ; O=C(Cl)c1ccc(Cl)s1 ; O=C(O)c1ccc(Cl)s1 ; O=C(NC[C@@H]1CN(c2ccc(N3CCOCC3=O)cc2)C(=O)O1)c1ccc(Cl)s1]
wherein the solvent used in both steps comprises water.

2. The process according to claim 1, wherein the compound of formula VII is condensed with a compound of formula VIII to prepare rivaroxaban of formula I.

3. The process according to claim 1, wherein the compound of formula VII is condensed with a compound of formula IX to prepare rivaroxaban of formula I.

4. The process according to claim 1, wherein the solvent used in the step of forming compound of formula VII is water.

5. The process according to claim 1, wherein the solvent used in the step of forming rivaroxaban is a mixture of water and one or more further solvents.

6. The process according to claim 5, wherein the or each further solvent is selected from the group consisting of acetone and dimethyl carbonate.

7. The process according to claim 1, wherein the first base is selected from the group consisting of a C1-C4 alkyl ammonia; mono, a di or tri CI-C4 alkyl amine; a mono, di or tri hydroxy C1-C4 alkyl amine; morpholine; thiomorpholine; piperidine; N,N-dimethylaniline; pyridine; a hydrazine and pyrrolidine.

8. The process according to claim 7, wherein the first base is aqueous methyl amine.

9. The process according to claim 1, wherein the second base is selected from the group consisting of an alkali or alkaline earth metal hydroxide; an alkoxide; a carbonate; a bicarbonate; and an organic base.

10. The process according to claim 9, wherein the second base is potassium carbonate.

11. The process according to claim 1, wherein the compound of formula VII is not isolated before the condensation step.

12. The process according to claim 1, wherein the compound of formula VI is prepared by cyclisation of a compound of formula V 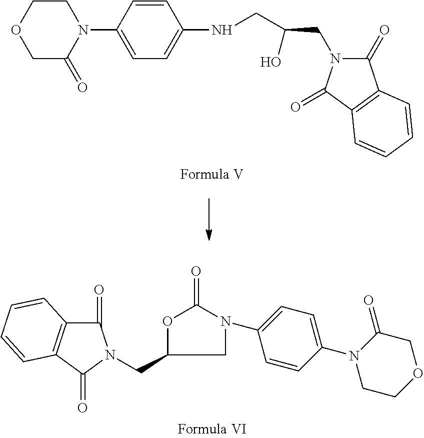 [SMILES: O=C1c2ccccc2C(=O)N1C[C@@H](O)CNc1ccc(N2CCOCC2=O)cc1 ; O=C1c2ccccc2C(=O)N1C[C@@H]1CN(c2ccc(N3CCOCC3=O)cc2)C(=O)O1]
in the presence of a carbonylating reagent and a base in a solvent which is a single solvent or a mixture of solvents.

13. The process according to claim 12, wherein the carbonylating reagent is selected from the group consisting of N,N′-carbonyl diimidazole, N,N′-carbonyl-di-1,2,3-benzotriazole, N,N′-carbonyl-di-1,2,4-triazole, disuccinimidyl carbonate dialkyl carbonates, phosgene, diphosgene, triphosgene, methyl chloroformate, benzyl chloroformate and phenylchloroformate.

14. The process according to claim 12, wherein the base in the cyclisation step is selected from one or more of an alkali or alkaline earth metal hydroxide; an alkoxide; a carbonate; a bicarbonate; and an organic base.

15. The process according to claim 12, wherein the solvent for the cyclisation step is selected from the group consisting of an alcohol; a ketone; a dialkyl carbonate; a diaryl carbonates; and water.

16. The process according to claim 12, wherein the cyclisation reaction is carried using dimethyl carbonate alone as the solvent, N,N′-carbonyldiimidazole as the carbonylating reagent and dimethylaminopyridine as the base.

17. The process according to claim 2, wherein the compound of formula VI is not isolated before being reacted to form Compound VII.

18. The process according to claim 12, wherein the compound of formula VI is isolated before being reacted to form Compound VII.

19. The process according to claim 1, wherein the compound of formula V is prepared by condensing a compound of formula III with (S)-Glycidyl Phthalimide (Formula IV); 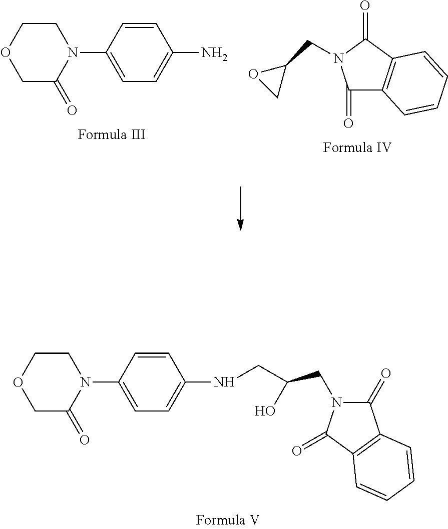 [SMILES: Nc1ccc(N2CCOCC2=O)cc1 ; O=C1c2ccccc2C(=O)N1C[C@H]1CO1 ; O=C1c2ccccc2C(=O)N1C[C@@H](O)CNc1ccc(N2CCOCC2=O)cc1]
in the presence of a solvent which is a single solvent or a or mixture of solvents to form the compound of formula V.

20. The process according to claim 19, wherein the solvent for the condensation step to form compound V is selected from the group consisting of a C₁-C₅ alcohol; a ketone; and water.

21. The process according to claim 19, wherein the condensation step to form compound V is carried out using water alone as the solvent and without base.

22. The process according to claim 19, wherein the compound of formula V is not isolated before being reacted to form compound VI.

23. The process according to claim 19, wherein the compound of formula V is isolated before being reacted to form compound VI.

24. The process according to claim 19, wherein the compound of formula III is prepared by reducing a compound of formula II 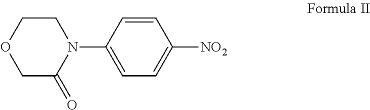 [SMILES: O=C1COCCN1c1ccc([N+](=O)[O-])cc1]
in the presence of a hydrogenation catalyst, and reagent and a solvent which is a single solvent or a or mixture of solvents to form the compound of formula III.

25. The process according to claim 24, wherein the solvent for the reduction is selected from the group consisting of a C₁-C₃ alcohol, an ester, an ether, a nitrile, tetrahydrofuran, water, a halogenated solvent, dimethylformamide, dimethyl sulfoxide, sulfolane, or a mixture thereof.

26. The process according to claim 24, wherein the hydrogenation catalyst for the reduction is selected from the group consisting of a noble metal catalyst which is preferably supported on a material selected from activated carbon, calcium carbonate, silicon dioxide, and triphenyl phosphine; a nickel-based catalyst selected from Raney nickel, and Urushibara nickel; zinc dust; palladium acetate; Iron; alumina; silica; calcium carbonate; barium sulphate; and a zeolite.

27. The process according to claim 24, wherein the reagent employed for reduction is selected from formic acid, ammonium chloride, ammonium formate, ammonia, an alkaline metal hydride, an alkali metal hydride, hydrochloric acid, sodium hydrosulfide and ammonium sulfide.

28. The process according to claim 24, wherein the reduction step involves the use of water alone as a solvent, Raney-Nickel as the catalyst and ammonium formate as the reagent.

29. The process according to claim 24, wherein the compound of formula III is not isolated before being reacted to form compound V.

30. The process according to claim 24, wherein the compound of formula III is isolated before being reacted to form compound V.

31-36. (canceled)
